(19) United States
(12) Patent Application Publication
(10) Pub. No.: US 2020/0388353 A1
(43) Pub. Date: Dec. 10, 2020

(54) AUTOMATIC ANNOTATION OF SIGNIFICANT INTERVALS OF GENOME

(71) Applicant: MOUNT SINAI GENOMICS, INC., Stamford, CT (US)

(72) Inventors: Joseph E. Barrows, White Plains, NY (US); Matthew Croken, New York, NY (US); Andrei Salomatov, New York, NY (US); Robert B. Calder, New York, NY (US); Andrew S. McLellan, Stamford, CT (US)

(21) Appl. No.: 16/893,223

(22) Filed: Jun. 4, 2020

Related U.S. Application Data
(60) Provisional application No. 62/858,293, filed on Jun. 6, 2019.

(51) Int. Cl. G16B 50/10; G16B 45/00; G16B 30/10; G16B 40/00; G16B 20/20

(52) U.S. Cl. CPC G16B 50/10; G16B 45/00; G16B 20/20; G16B 40/00; G16B 30/10

(57) ABSTRACT

A target interval builder system may generate genomic feature tracks based on gene identifiers provided by users. The system may build the genomic feature tracks based on data obtained from various biomarker data servers. An example target interval building process may include receiving one or more identifiers. The system may retrieve various sets of genomic coordinates from different data sources. The sets of genomic coordinates may include information of various genomic intervals that are identified from the gene identifiers. The system may standardize the sets of genomic coordinates retrieved to generate a standardized set of genomic coordinates that includes genomic coordinates of target intervals. The genomic feature tracks may be generated from the standardized genomic coordinates and related annotation data. The system may annotate the intervals with information such as standardized gene symbols, gene identifiers, and sources. The system may provide sequencing performance and mappability analyses for the target intervals.

### CROSS-REFERENCE TO RELATED APPLICATION

The present application claims the benefit of U.S. Provisional Patent Application No. 62/858,293 filed on Jun. 6, 2019, which is hereby incorporated by reference in its entirety.

### BACKGROUND

Post-Sanger sequencing technologies, such as next-generation sequencing (NGS), have become increasingly ubiquitous in biological and pharmaceutical research. NGS is a diverse collection of sequencing technologies that offer a vastly increased scale of sequencing at significantly reduced cost when compared with Sanger sequencing. In NGS, a massive number of DNA fragments are sequenced simultaneously. Prior to NGS sequencing, the sample preparation process typically involves library preparation to generate a collection of DNA fragments, with appropriate DNA adapters added, for sequencing. Fragmented libraries may be subjected to hybridization with custom-designed oligonucleotide probes specific to regions of interest (hybrid capture). When performing hybrid capture, non-specific unbound regions are washed away, thereby enriching the regions of interest for sequencing purposes. In a clinical study, or for other research, the precise regions of interest of genes, mutations, and/or phenotypes are often difficult to determine, thus leading to costly and labor-intensive processes to select and design oligonucleotide probes. Also, it is desirable to apply a consistent, documented approach to designing the probes.

### SUMMARY

Hybrid capture-based target enrichment uses custom-designed oligonucleotide probes to capture specific regions of the genome using massively parallel sequencing (e.g., NGS). In one embodiment, a target interval builder (TIB) system selects regions of the genome that are clinically relevant for analysis. The TIB system retrieves information from multiple genetic data sources and combines the information into a standard format. The information may include versioning information for the corresponding sources for the genomic intervals and variants. The TIB system allows a user to search the regions based on a target gene list, thereby allowing a user to access the information needed for individual genes. The TIB system uses multiple tools to evaluate regions as problematic or amenable to NGS analysis. Identification of problems expedites review of data by NGS analysis.

The TIB system may provide a user interface or command line input configured to allow users to provide input, for example, by uploading files. In one embodiment, users may provide a text file containing HGNC (HUGO Gene Nomenclature Committee) gene identifiers and optionally Browser Extensible Data (BED) files of regions to include or exclude via the user interface. Based on the user's input, the TIB system generates annotated genomic coordinates for each gene, associated transcripts, and associated variants and present the generated or collected data in the form of genomic feature tracks. The TIB system builds the annotated coordinates based on data obtained from various biomarker data servers.

By way of example, a target interval building process may include receiving one or more gene identifiers from a user. The process may also include retrieving a first set of genomic coordinates from a first biomarker data server. The retrieved information may include information of the first set of genomic coordinates, one or more genes, variants, transcripts, phenotypes, introns, untranslated intervals, and/or intergenic intervals that are identified from the one or more gene identifiers. The retrieved information may be in a first unstandardized format. The process may further include retrieving a second set of genomic coordinates from a second biomarker data server. The retrieved information may include information of the second set of genomic coordinates, one or more genes, variants, transcripts, phenotypes, introns, untranslated intervals, and/or intergenic intervals that are identified from the one or more gene identifiers. The retrieved information may be in a second unstandardized format. The process may further include generating standardized genomic feature tracks from at least the first and second genomic coordinates and associated annotations that may be in unstandardized formats. The standardized genomic feature tracks may include genomic coordinates, genes, variants, transcripts, phenotypes, introns, untranslated intervals, and/or intergenic coordinates of target intervals derived from the genomic coordinates in the first set and the second set. The combined genomic feature tracks may be in a standardized format. The process may further include providing the standardized genomic feature tracks to the user.

The biomarker data servers that may be used for retrieval of data may include the University of California, Santa Cruz (UCSC) Genome Browser, the HUGO Gene Nomenclature Committee (HGNC; via genenames.org), the European Bioinformatics Institute and the Wellcome Trust Sanger Institute Ensembl Genome Browser, National Center for Biotechnology Information (NCBI) ClinVar, and the Qiagen Human Gene Mutation Database (HGMD) databases. Other suitable data sources may also be used. The TIB system may version the files retrieved from those data sources using a standardized naming convention.

When designing a set of new targets, the TIB system searches sets of genomic coordinates using the list of gene identifiers provided at runtime. In one embodiment, the TIB system may filter genomic coordinates to include only those that contain protein-coding transcripts unless no protein-coding transcripts are available. Other output options may also be available. Additionally, or alternatively, the TIB system may split the resulting set of genomic coordinates. For example, one split set of genomic coordinates may include untranslated regions (UTRs) and introns while another split set of genomic coordinates may exclude those regions. The TIB system may also analyze the sets of genomic coordinates to gather metrics for parameters that may impact NGS performance, such as segmental duplications, repeats, and uniqueness of read alignment. The TIB system provides the outputs to the user, for example, by depositing the output files in an output location, such as in a cloud storage that is accessible by the user.

In generating standardized genomic feature tracks, the TIB system annotates the target intervals with, for example, the HGNC approved gene symbols, HGNC identifiers (or other gene symbols), and sources. Other annotations may include NCBI RefSeq transcript identifiers, exon numbers, HGMD accessions, Ensembl transcript identifiers, UCSC Genome Browser timestamps, Human Gene Variation Society (HGVS) variants, ClinVar identifiers, and types of region (intron, CDS, UTR, etc.). The TIB system annotates genomic intervals to facilitate the design and filtering of targeted NGS capture libraries. Standardized annotation retains interval provenance throughout library design. Poorly performing intervals may be intersected with genes, variants, and regions to expedite analysis. NGS data can be subset for targeted analysis by filtering annotated intervals against a custom list of HGNC identifiers.

### BRIEF DESCRIPTION OF THE DRAWINGS

FIG. 1 illustrates a diagram of a system environment of an example target interval builder system, in accordance with an embodiment.

FIG. 2 is a block diagram of an architecture of an example target interval builder system, in accordance with an embodiment.

FIG. 3 is an example front-end graphical user interface, in accordance with an embodiment.

FIG. 4 is a flowchart depicting an example process for target genomic interval information based on a gene search term, in accordance with an embodiment.

FIG. 5 is a flowchart depicting an example process that generates genomic feature tracks, in accordance with an embodiment.

FIG. 6 is a flowchart depicting an example process of performing amenability analyses for massively parallel sequencing, in accordance with an embodiment.

FIG. 7 is a flowchart depicting an example process of performing mappability analyses for massively parallel sequencing, in accordance with an embodiment.

FIGS. 8A and 8B are conceptual diagrams illustrating various examples of standardized genomic feature tracks, in accordance with some embodiments.

FIG. 9 is a flowchart depicting an example process of generating standardized genomic feature tracks, in accordance with an embodiment.

FIG. 10 is a flowchart depicting an example process of performing biological sample analyses that involve massively parallel sequencing, in accordance with an embodiment.

FIG. 11 is a block diagram of an example computing device, in accordance with an embodiment.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

The figures (FIGs.) and the following description relate to preferred embodiments by way of illustration only. One of skill in the art may recognize alternative embodiments of the structures and methods disclosed herein as viable alternatives that may be employed without departing from the principles of what is disclosed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Example System Environment

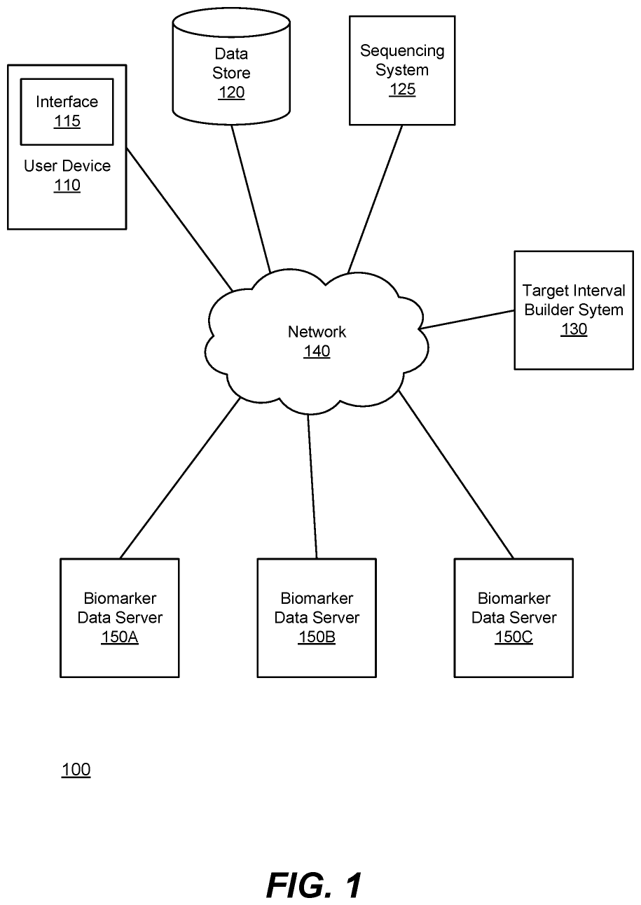

FIG. 1 illustrates a diagram of a system environment 100 of an example target interval builder system, in accordance with an embodiment. The system environment 100 shown in FIG. 1 includes one or more client devices 110, a data store 120, a sequencing system 125, a target interval builder (TIB) system 130, a network 140, and one or more biomarker data servers 150A, 150B, 150C (collectively as biomarker data servers 150 or a biomarker data server 150). In various embodiments, the system environment 100 may include fewer or additional components. The system environment 100 may also include different components.

The client devices 110 are one or more computing devices capable of receiving user input as well as transmitting and/or receiving data via a network 140. Example computing devices include desktop computers, laptop computers, personal digital assistants (PDAs), smartphones, tablets, wearable electronic devices (e.g., smartwatches), smart household appliance (e.g., smart televisions, smart speakers, smart home hubs), Internet of Things (IoT) devices or other suitable electronic devices. A client device 110 communicates to other components via the network 140. In one embodiment, a client device 110 executes an application that launches a graphical user interface (GUI) for a user of the client device 110 to interact with the TIB system 130. The GUI may be an example of a user interface 115. A client device 110 may also execute a web browser application such as a web form to enable interactions between the client device 110 and the TIB system 130 via the network 140. In another embodiment, the user interface 115 may take the form of a software application published by the TIB system 130 and installed on the user device 110. In yet another embodiment, a client device 110 interacts with the TIB system 130 through an application programming interface (API).

The data store 120 may be one or more computing devices that include memories or other storage media for storing genomic feature tracks and other suitable files and data. The files may be provided by the client device 110 or the TIB system 130. The data store 120 may be a network-based storage server (e.g., a cloud server). The data store 120 may be part of the TIB system 130 or may be a third-party storage system such as AMAZON AWS include AMAZON S3, DROPBOX, RACKSPACE CLOUD FILES, AZURE BLOB STORAGE, GOOGLE CLOUD STORAGE, etc. In some cases, the data store 120 also may be referred to as a cloud storage server 120.

The sequencing system 125 may include various sequencing machines to extract genetic data from biological samples (e.g., saliva, blood, hairs, tissues) of individuals, who may be referred to as subjects or patients. The sequencing system 125 may use various nucleotide processing techniques such as amplification and sequencing. Amplification may include using polymerase chain reaction (PCR) to amplify segments of nucleotide samples. Sequencing may include sequencing of deoxyribonucleic acid (DNA), ribonucleic acid (RNA) sequencing, etc. Suitable sequencing techniques may include Sanger sequencing and massively parallel sequencing such as various next-generation sequencing (NGS) techniques including whole genome sequencing, pyrosequencing, sequencing by synthesis, sequencing by ligation, and ion semiconductor sequencing. The sequencing system 125 performs sequencing of the biological samples and determines the nucleotide sequences of the individuals. The sequencing system 125 generates data of the sequences of individuals' genome or part of the genome based on the sequencing results. The data may include data sequenced from DNA or RNA and may include base pairs from coding and/or noncoding regions of genome.

The target interval builder system 130 may include one or more computing devices that generate one or more biomarker coordinates (e.g., genomic coordinates) and perform analysis of sequence data provided by the sequencing system 125. The target interval builder system 130 may be referred to as a TIB system or a computing server. The TIB system 130 may receive, from a user device 110, an input that includes one or more biomarker search terms. The search terms may be expressed as gene identifiers or other suitable search terms. In response, the TIB system 130 may automatically create one or more genomic feature tracks that include relevant information related to the biomarker search terms. The genomic feature tracks, which are generated in certain specific formats, may be a collection of data that may include target genomic coordinates or intervals of interest and may also include information related to the coordinates or intervals such as HGNC identifier, MIM identifier, transcript identifier, exon, accession, etc. The genomic feature tracks may be presented as raw data or may be formatted into a common file type such as a BED file. The genomic feature tracks may be transmitted directly to the user device 110 via the network 140 or be transferred to data store 120, which may be accessible by user device 110.

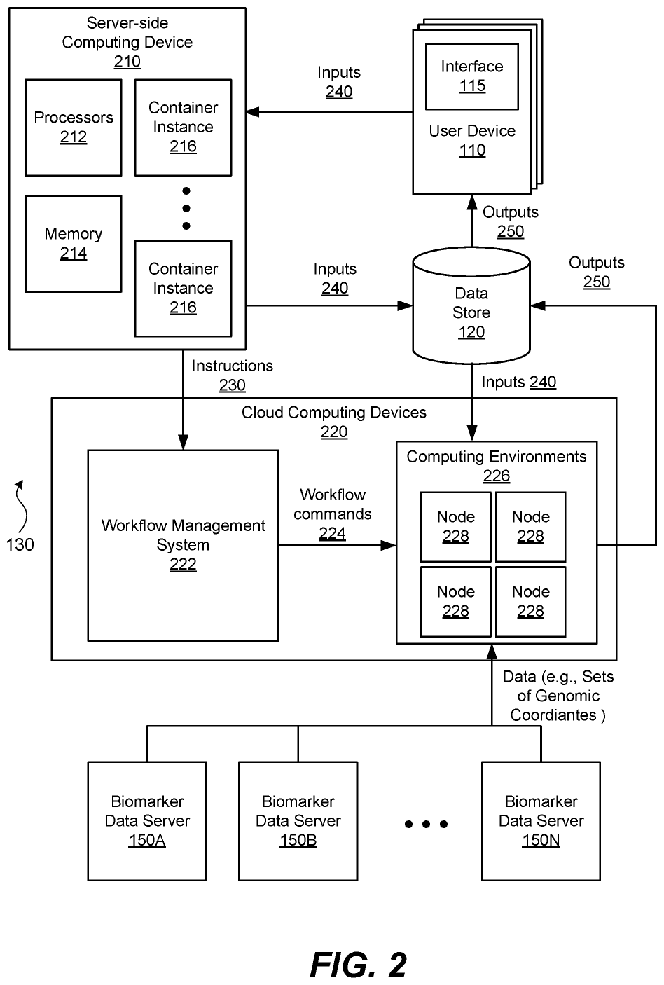

In various embodiments, the TIB system 130 may take different forms. The TIB system 130 may be a server computer that includes software and one or more processors to execute code instructions to perform various processes described herein. The TIB system 130 may also be a pool of computing devices that may be located at the same geographical location (e.g., a server room) or be distributed geographically (e.g., cloud computing, distributed computing, or in a virtual server network). An example structure and arrangement of an embodiment of the TIB system 130 is discussed in further detail with reference to FIG. 2.

The TIB system 130 may use various systems in providing the target interval building functionality. Some examples of systems that may be used by the TIB system 130 include AWS BATCH, EC2, EBS, and S3; the Broad Institute CROMWELL; BEDTOOLS; BCFTOOLS; DOCKER; BIOMART; NGINX, DJANGO, and includes custom-developed scripts written in the Broad Institute WORKFLOW DESCRIPTION LANGUAGE (WDL), PYTHON, BASH, R and MYSQL. Multiple Docker images, stored in AWS ECR, may be used to perform the target interval building functionality.

The communications between the client devices 110, the data store 120, the TIB system 130 may be transmitted via a network 140, for example, via the Internet. The network 140 provides connections to the components of the system 100 through one or more sub-networks, which may include any combination of local area and/or wide area networks, using both wired and/or wireless communication systems. In one embodiment, a network 140 uses standard communications technologies and/or protocols. For example, a network 140 may include communication links using technologies such as Ethernet, 802.11, worldwide interoperability for microwave access (WiMAX), 3G, 4G, Long Term Evolution (LTE), 5G, code division multiple access (CDMA), digital subscriber line (DSL), etc. Examples of network protocols used for communicating via the network 140 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over a network 140 may be represented using any suitable format, such as hypertext markup language (HTML), extensible markup language (XML), or JSON. In some embodiments, all or some of the communication links of a network 140 may be encrypted using any suitable technique or techniques such as secure sockets layer (SSL), transport layer security (TLS), virtual private networks (VPNs), Internet Protocol security (IPsec), etc. The network 140 also includes links and packet switching networks such as the Internet.

The biomarker data servers 150 may be one or more data servers that provide information regarding various biomarkers. One of the biomarker data servers 150 may be part of the TIB system 130 and other biomarker data servers 150 may be third party databases or data providers. Suitable data servers may include genomic coordinate and sequence sources that may provide data regarding sequences of genomes for humans and other organisms, a sequence version source that provide data regarding different sequence versions in various genetic loci, a gene name source that may provide nomenclature of genes, a mutation data source that may provide data regarding common mutations, and variant-phenotype relation database that may provide data regarding the association among a phenotype and one or more genetic loci or single nucleotide polymorphism (SNP). Example biomarker data servers 150 may include University of California, Santa Cruz (UCSC) Genome Browser, the HUGO Gene Nomenclature Committee (HGNC; via genenames.org), the European Bioinformatics Institute and the Wellcome Trust Sanger Institute Ensembl Genome Browser, National Center for Biotechnology Information (NCBI) ClinVar, and the Qiagen Human Gene Mutation Database (HGMD). Other biomarker data servers 150 may include databases that store clinical study data, scientific papers, medical records, and suitable university databases.

### Example Target Interval Builder System Architecture

FIG. 2 is a block diagram illustrating the architecture of an example TIB system 130, in accordance with an embodiment. The example TIB system 130 shown may include one or more computers such as one or more server-side computing devices 210 and cloud computing devices 220. The server-side computing device 210 and the cloud computing devices 200 each may include one or more processors 212 and memory 214. The memory 214 may store computer code that includes instructions 230. The instructions 230, when executed by one or more processors 212 (in a computing device 210 or 220), cause the processors 212 to perform one or more processes described herein, such as one or more workflows defined by instructions 230. In one embodiment, the server-side computing device 210 and the cloud computing devices 220 may be implemented in a distributed manner. For example, the server-side computing device 210 may communicate with the cloud computing devices 220 via the network 140. The cloud computing devices 220 may include multiple computers operated in a distributed fashion.

While an example architecture of the TIB system 130 is shown in FIG. 2, the TIB system 130 may take other forms. For example, in another embodiment, instead of implementing cloud computing devices 220, the TIB system 130 may take the form of a non-cloud server. The computing devices 220 may be one of the on-site servers that communicate with the server-side computing device 210 locally. In yet another embodiment, the TIB system 130 may take the form of a personal computer that executes code instructions directly instead of using any additional computing devices 220. Other suitable implementations are also possible.

In the example shown in FIG. 2, the TIB system 130 is implemented as a cloud computing system. A server-side computing device 210 may include instructions stored in memory 214 to cause the processors 212 to generate one or more instances of virtualization of user spaces. Each virtualization instance may be referred to as a container instance 216, which may be a virtual machine, a Docker, a virtual private server, a virtual kernel, or another suitable virtualization instance. The container instances 216 may independently communicate with the cloud computing devices 220 using instructions 230 to perform one or more processes in various instances. In one embodiment, various container instances 216 may be created for performing different roles. For example, a first container instance 216 may specialize in communicating with user devices 110. A second container instance 216 may specialize in providing and retrieving data and other information from data store 120. A third container instance 216 may specialize in controlling one or more workflow processes by providing instructions 230 to the cloud computing devices 220.

The cloud computing devices 220 include a workflow management system 222 that executes one or more workflow processes. The workflow management system 222 may be a cloud computing system that executes the instructions 230, which may describe a job scheduler or otherwise generally the steps in a workflow process. In one embodiment, the workflow management system 222 is a CROMWELL system, although in various embodiments other suitable workflow management systems may also be used. Instructions 230 may be in any suitable format such as in Workflow Description Language (WDL). Based on instructions 230, the workflow management system 222 sends workflow commands 224 to computing environments 226 to execute one or more steps. In one embodiment, the computing environments 226 include one or more nodes 228 that are used as separate computing environments for parallel executions of different programs in various program languages. For example, the nodes 228 may execute programs in BASH, PYTHON, MYSQL, R, etc. through the nodes 228 in the computing environments 226. The computing environments 226 are in communication with the data store 120 and one or more biomarker data servers 150.

A request for a workflow process may be initiated from a user device 110 in response to the user device 110 uploading one or more inputs 240, which may include one or more biomarker search terms (e.g., gene identifiers) and may also include user-specific genomic coordinates that are intended to be included or excluded in the results. The server-side computing device 210 receives the inputs 240 and provides instructions 230 to the cloud computing devices 220 to execute one or more workflows. The server-side computing device 210 may also upload the inputs 240 to the data store 120. The workflow management system 222 provides workflow commands 224 to the computing environments 226. The computing environments 226 execute a program based on the workflow commands 224. For example, a node 228 may retrieve the inputs 240 from the data store 120 or directly from the server-side computing device 210. The node 228, based on the program and workflow commands 224, also communicates with one or more biomarker data servers 150 (e.g., via the APIs of the biomarker data servers 150) to retrieve relevant information from those servers 150. The node 228 also performs other processing of the data and information to generate outputs 250, which may include one or more annotated sets of genomic coordinates in the form of genomic feature tracks that contain information related to genomic coordinates, genes, variants, transcripts, phenotypes, intron, coding, untranslated, and/or intergenic areas of interest in the genome that may be related to the genes identified in the inputs 240. The annotated coordinates may include the type of regions, source of information, and additional gene identifiers that may be relevant to genes specified in the inputs 240. The outputs 250 may be saved in the data store 120, which may be accessible by user device 110, or may be directly provided to the user device 110. The user device 110 may display the information in genomic feature tracks in the interface 115.

### Example Input Portals

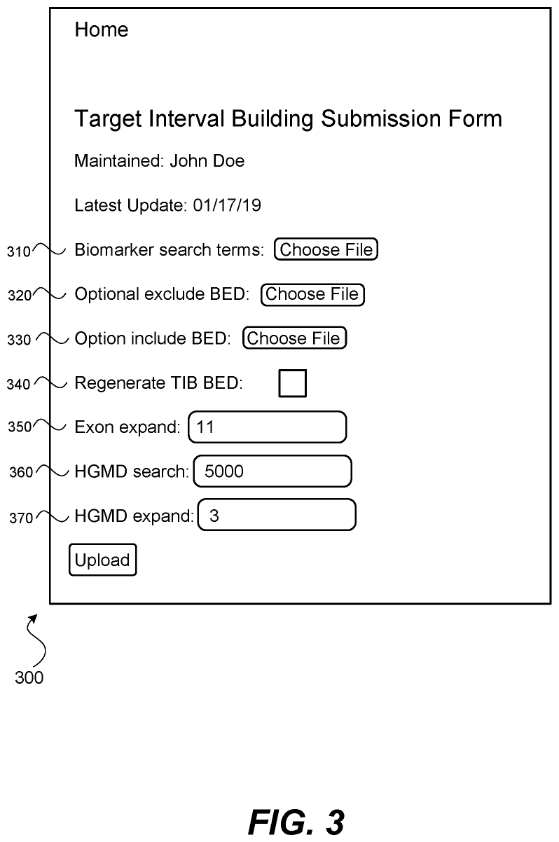

FIG. 3 is an example front-end graphical user interface (GUI) 300 that may be displayed at interface 115 of a user device 110, in accordance with an embodiment. The GUI 300 is an input portal that allows users to enter or upload various inputs. The GUI 300 may include versioning information such as the system version, update date, and system maintainer. The GUI 300 allows users to upload input files or otherwise enter (e.g., by directly typing at the GUI 300) input information for the TIB system 130 to initialize one or more workflow processes to generate an output that includes one or more biomarker coordinates. Although a GUI which is shown as a web form is shown in FIG. 3, the TIB system 130 may also include other types of interfaces that communicate with users, such as a command-line interface, an API, etc.

The inputs for initializing a workflow process may include one or more biomarker search terms 310. The biomarker search terms 310 may specify the genes for which a user intends to generate intervals and information regarding the intervals. The biomarker search terms 310 may be uploaded by a user as a file (e.g., a text file) that arranges search terms one term per line. In different embodiments, the TIB system 130 may support biomarker search terms in various suitable formats. In one embodiment, the biomarker search terms are in a standardized gene identifier format. For example, the gene identifiers are in the HGNC gene nomenclature format. Other suitable gene identifier formats may also be used. In one embodiment, the TIB system 130 may allow biomarker search terms to be in a looser gene name format instead of using a standardized gene identifier. For example, the gene search terms may be in the form of gene identifiers or gene name. In another embodiment, the biomarker search terms may be in a natural language phrase such as “a gene that causes cystic fibrosis.” In another embodiment, the biomarker search terms may include protein identifier, protein name, transcript identifier, transcript name, variant identifier, variant name, variant pathogenicity, coding/intronic/intergenic/untranslated/promoter/homologous/repetitive/high AT or GC content/pseudogenes/other regions of interest, as well as raw genomic coordinates.

In addition to the biomarker search terms 310, the inputs may also include genomic coordinates that should be included or excluded in the outputs. The genomic coordinates may be directly inputted at the GUI 300 or be included in one or more genomic feature tracks that are formatted as tab-delimited text files such as BED (Browser Extensible Data) files that are uploaded to the GUI 300. A user may upload an “include” BED file 330 that delineates coordinates that should be added in the outputs. For example, in some cases, the user may intend to include intronic regions of a gene in all outputs, such as those that may normally not include intronic regions. The user may also upload an “exclude” BED file 320 that delineates coordinates that should be removed from the outputs. For example, in some cases, a user may be aware of genomic regions that do not perform well with a massively parallel sequencing and may intend to exclude those regions form the outputs. In various cases, more than one “include” and “exclude” BED files 320 and 330 may be uploaded. In other cases, no coordinates are specified in an input.

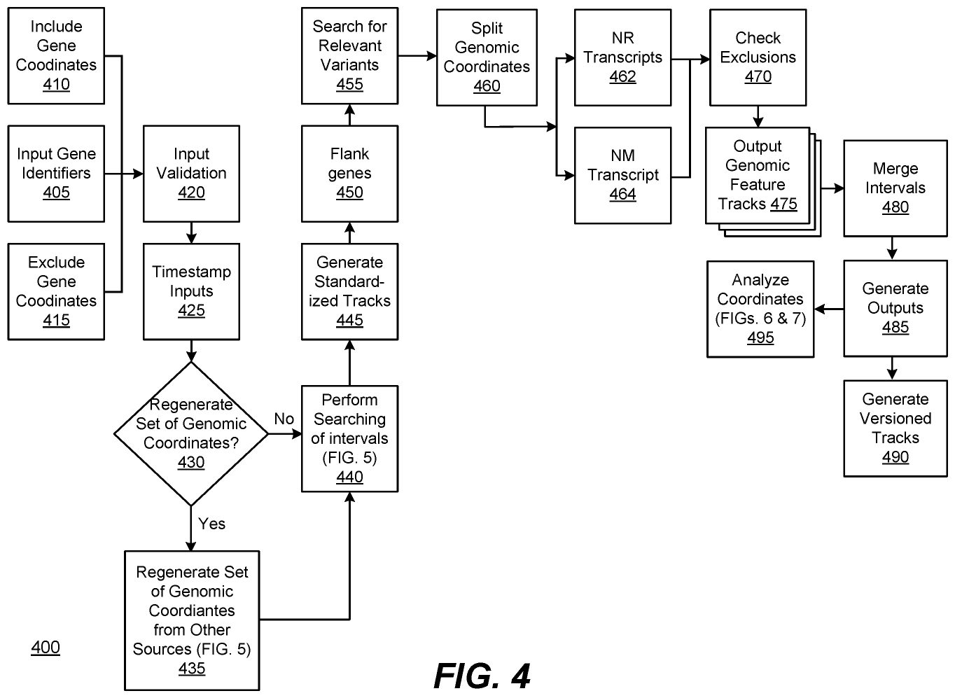

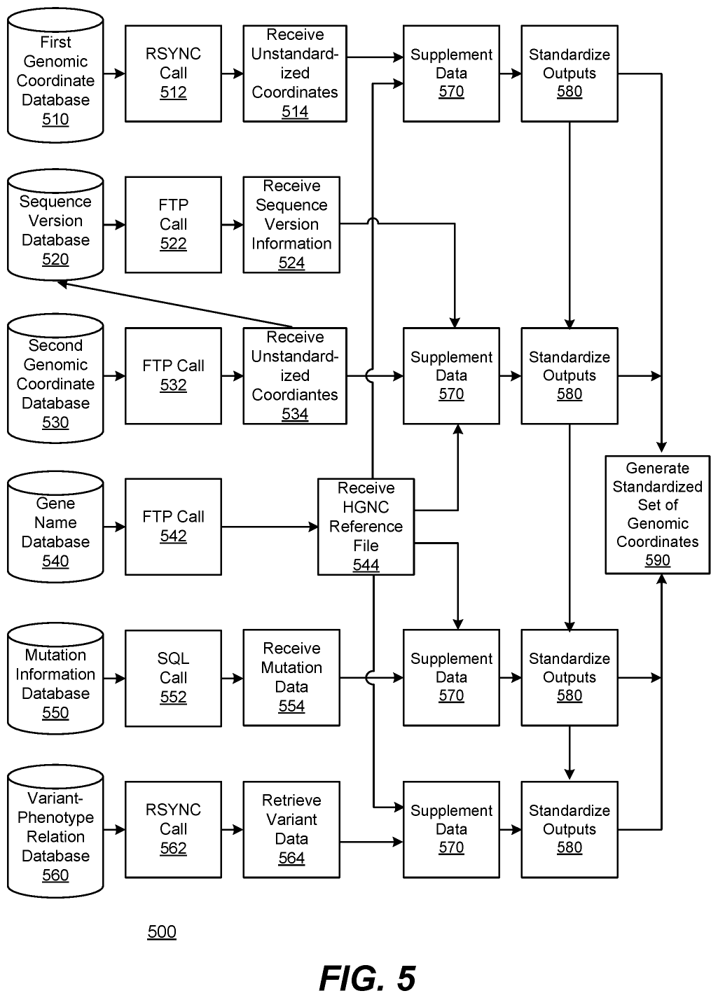

The option for regenerating TIB BED 340 allows the TIB system 130 to regenerate the BED file (or another suitable form of a collection of biomarker coordinates) from source databases. For example, a user may specify a gene identifier in the input. By selecting the generating TIB BED 340 option, the TIB system 130 regenerates the BED files regardless of whether the same BED files were generated before. Depending on the user's choice, the TIB system 130 may generate a new reference BED file or use a previously constructed BED file. The processing of regenerating source BED files will be discussed in detail with reference to FIGS. 4 and 5.

The GUI 300 may also accept other preference choices as part of the input. For example, the “exon expand” field 350 allows users to specify the number of bases that will be added to both sides of the coding DNA sequences. The “HGMD search” field 360 allows users to specify the number of bases on either side of the genes that should be searched for potential HGMD variants that may be unlabeled or mislabeled. The “HGMD expand” field 370 allows users to specify the number of bases that will be added to both sides of an HGMD or another variant to be included in the outputs.

While GUI 300 shows several example input fields, other inputs are also suitable in various embodiments. An embodiment may include additional or different input fields. Other embodiments may also include fewer input fields than GUI 300. The GUI 300 may be implemented as part of a web application (e.g., JavaScript application), a mobile application, a desktop software application, or another suitable application.

While a GUI 300 is shown in FIG. 3, in some embodiments, the TIB system 130 may be accessed from a user using command line directly or command line window without a GUI. For example, in some embodiments, the TIB system 130 may communicate with a user using LINUX commands such as Secure Socket Shell (SSH).

### Example Workflow Process

FIG. 4 is a flowchart depicting an example process 400 for target genomic interval information based on one or more biomarker search terms such as gene identifiers, in accordance with an embodiment. In one embodiment, the process 400 may correspond to one of the workflow processes that may be executed by the workflow management system 222 in response to a user inputting one or more biomarker search terms through GUI 300. The process 400 may be executed by the TIB system 130. The process 400 generates one or more biomarker coordinates as outputs.

The TIB system 130 may receive a search request that includes one or more biomarker search terms such as input gene identifiers 405. The input gene identifiers may correspond to the biomarker search terms 310 inputted by a user through GUI 300. The inputs received by the TIB system 130 may also encompass the “include” genomic coordinates 410 and the “exclude” genomic coordinates 415 that may be included in one or more BED files 320 and 330. Upon receiving the inputs, the TIB system 130 validates 420 the inputs by checking the formats of the inputs 405, 410, and 415 to determine whether the values are in the correct formats that are supported by the TIB system 130. If one or more inputs are in the wrong formats or are not recognizable, the TIB system 130 may provide an error message in GUI 300 and reject the search request. If the inputs are validated, the TIB system 130 may generate a unique identifier for the search request to allow the outputs of the search request to be found in data store 120.

The TIB system 130 may perform several pre-processing operations of the inputs before the biomarker data servers are accessed. For example, the TIB system 130 may timestamp 425 the inputs 405, 410, and 415 by associating a particular instance of the process 400 with a date and time. The TIB system 130 may generate the unique identifier of the instance of the process 400 based on the timestamp. The TIB system 130 may also determine 430 whether the user has requested the regeneration of reference genomic feature tracks. For example, in the GUI 300 in FIG. 3, if a user selects the option for generating TIB BED 340, the TIB system 130 will generate 435 the reference set of genomic feature tracks. The regeneration of the reference genomic feature tracks will be discussed in further detail with reference to FIG. 5.

For gene identifiers that have been not searched before or that are requested to be re-generated, the TIB system 130 performs 440 searches of the genomic intervals based on the gene identifiers specified in the inputs. If regeneration of the reference genomic feature tracks is not requested, the TIB system 130 may search a data store, such as the data store 120, to locate the reference genomic feature tracks that are previously saved. To conduct the search 440, the TIB system 130 may use the HGNC gene identifiers specified in or derived from the inputs to conduct the search. Based on the gene identifiers 405, the TIB system 130 searches the reference set of annotated genomic coordinates for the genomic feature coordinates that match or that are relevant to the gene identifier 405. The determined coordinates may be genomic coordinates that are found on top of the include genomic coordinates 410.

By way of example, in one embodiment, the TIB system 130 may retrieve a first set of genomic coordinates from a first biomarker data server 150. The first set of genomic coordinates may be associated with information of a first set of one or more genomic intervals that are identified from one or more gene identifiers and certain annotation information. The information of the retrieved first set of genomic coordinates may be in a first unstandardized format. The TIB system 130 may also retrieve a second set of genomic coordinates from a second biomarker data server 150. The second set of genomic coordinates may be associated with information of a second set of one or more genomic intervals that are identified from one or more gene identifiers. The information of the retrieved second set of genomic coordinates may be in a second unstandardized format. The TIB system 130 may also generate 445 standardized genomic feature tracks that include standardized sets of genomic coordinates. Genomic feature tracks may be standardized when data such as genomic coordinates from various sources are put into a standardized format and/or notation. The genomic feature tracks may be formatted in a suitable format such as in the BED file format. In one embodiment, standardized genomic feature tracks may be generated from at least the first and second sets of genomic coordinates. The standardized genomic feature tracks may include genomic coordinates of target intervals that are derived from the genomic intervals in the first set and the second set. The genomic coordinates in the standardized genomic feature tracks may be in a standardized format. The search of the gene identifiers and generation of standardized genomic feature tracks will be discussed in further detail with reference to FIG. 5.

In generating one or more sets of genomic coordinates, standardized or not, the TIB system 130 may include additional genomic intervals. For example, the TIB system 130 may receive one or more include genomic intervals that are intended to be included by the user and that are specified in the “include” genomic coordinates 410. The TIB system 130 may identify one or more additional genomic intervals that match the include genomic intervals but are not identified by the gene identifiers. The TIB system 130 may add the identified additional genomic intervals to the standardized genomic feature tracks.

The TIB system 130 flanks 450 the genomic intervals identified based on the input gene identifiers 405. After the genomic coordinates that are related to the gene identifiers 405 are determined, the TIB system 130 may flank each genomic interval by extending both sides of the interval with additional bases at each end of the genomic interval (e.g., 200 bases on each side) to generate a flanked target genomic interval. The genomic interval may be selected from any set of genomic intervals that are retrieved from one or more biomarker data server 150. The TIB system 130 searches 455 for disease-related or other-phenotype-related variants from one or more biomarker data servers 150 for both the intervals indicated by the genomic coordinates and the intervals extended from the flanking 450. The biomarker data servers 150 used to search for variants may be any suitable databases such as the HGMD. For example, the TIB system 130 searches for common SNPs, indels, or other mutations that are associated with any diseases or other phenotypes in any of the identified or extended intervals. The TIB system 130 incorporates the retrieved variant information in the standardized genomic feature tracks.

The TIB system 130 splits 460 the sets of genomic coordinates into multiple subsets. For example, the TIB system 130 may classify the genomic intervals included in the sets of genomic coordinates based on the coding or non-coding regions. In one embodiment, the TIB system 130 splits 460 the sets of genomic coordinates into NR transcripts 462 and NM transcripts 464. Non-coding genomic coordinates, such as NR transcripts 462, may include genomic coordinates that transcribe non-coding RNAs (ncRNAs). Coding genomic coordinates, such as NM transcripts 464, may include genomic coordinates that transcribe messenger RNAs (mRNAs). In one embodiment, the NM transcripts 464 may be preferred because those are protein-coding genomic coordinates. For example, in one case, if no protein-coding genomic coordinates are available, other genomic coordinates such as the NR transcripts 462 may be generated instead. In one embodiment, if NM transcripts 464 genomic coordinates are available, the NR transcripts 462, which include non-coding coordinates, will be removed from the final output. In one case, NR transcripts 462 are only retained when there is no NM transcript 464 present. However, in another embodiment, no such removal of the non-coding coordinates is performed.

The TIB system 130 checks 470 exclusions by comparing the genomic coordinates in the generated genomic feature tracks to the “exclude” genomic coordinates 415. For example, the TIB system 130 may receive one or more exclude genomic intervals that are intended to be excluded by the user and that are specified by the “exclude” genomic coordinates 415. The TIB system 130 identifies, in a set of genomic coordinates included in the standardized genomic feature tracks, one or more target genomic intervals that match the exclude genomic intervals. The TIB system 130 removes the identified target intervals from the genomic feature tracks and provides information in the genomic feature tracks on which genomic coordinates are removed based on user's requests.

The TIB system 130 outputs 475 different types of genomic feature tracks based on different queries. For example, a first type of genomic feature tracks may include only the coding intervals. A second example type of genomic feature tracks may include the coding intervals and untranslated regions (UTR). This type of genomic feature tracks may be generated by combining the NR transcripts 462 with the NM transcripts 464. A third example type of genomic feature tracks may include the coding intervals, the UTR, and introns. Other types of genomic feature tracks, which may include additional or fewer details, are also possible in various embodiments. For various types of sets of genomic feature tracks, information related to the variants and other coordinates of interest located in any of the intervals (e.g., coding, UTR, introns, etc.) may be included in the genomic feature tracks.

For each set of genomic feature tracks generated, the TIB system 130 may merge 480 the identified intervals in the genomic feature tracks. For example, the TIB system 130 may sort the genomic intervals based on the positions of the intervals in the genome. The TIB system 130 may identify multiple overlapped genomic intervals. The overlapped genomic intervals may be included in sets of genomic coordinates that are retrieved from different biomarker data servers 150. The TIB system 130 merges the overlapping intervals into a larger interval, which may be referred to as a merged interval. The merged interval may be one of the target intervals included in one of the standardized genomic feature tracks.

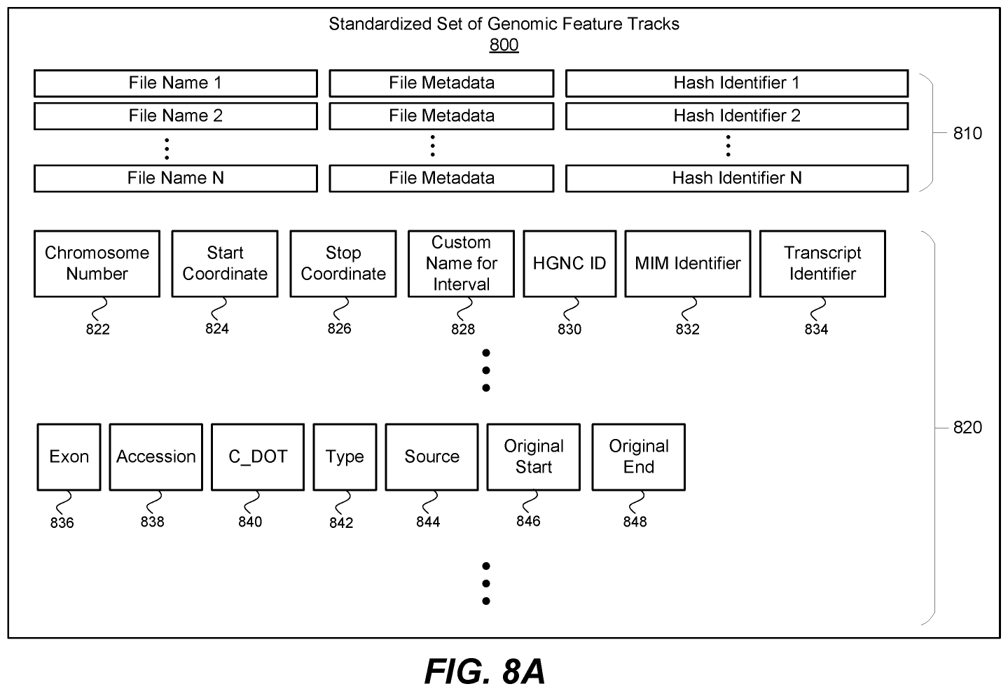

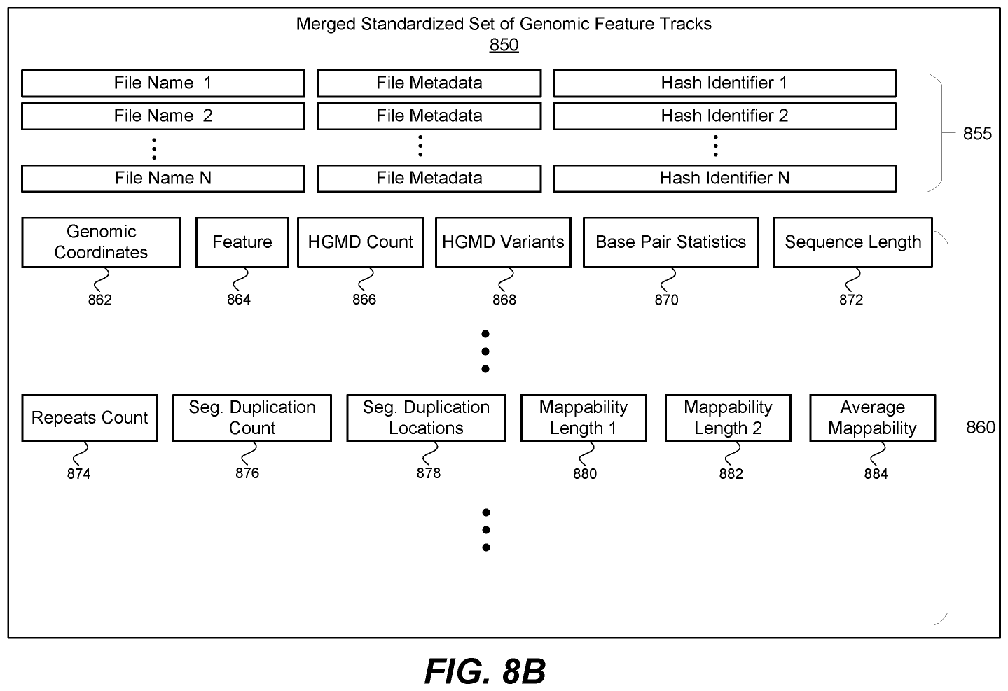

The TIB system 130 generates 485 the outputs. The outputs may be one or more sets of genomic feature tracks that are generated based on user's specification. The output genomic feature tracks may include annotated genomic intervals covering coding regions and various areas of interest that are related to the genes identified in the input gene identifiers 405. The annotated intervals may be expressed in genomic coordinates that are standardized. The annotated intervals may include information such as types of region, sources, additional identifiers as appropriate, and other suitable information that will be discussed in further detail with reference to FIGS. 8A and 8B. If users request non-coding regions, an additional set of genomic feature tracks that also cover UTRs and/or introns may also be generated. The inclusion or exclusion of non-coding intervals may also be gene-locus specific. For example, a user may specify that non-coding regions related to a first gene identifier should be included in the genomic feature tracks while those related to a second gene identifier should be removed from the genomic feature tracks. Also, the output genomic feature tracks may be merged or unmerged. In unmerged genomic feature tracks, the data includes intervals of features related to the searched gene identifiers 405 and the “include” genomic coordinates 410 with the “exclude” genomic coordinates 415 removed. In a merged set of genomic feature tracks, the data includes consolidated overlapping intervals annotated with statistics to predict the performance of NGS in these regions.

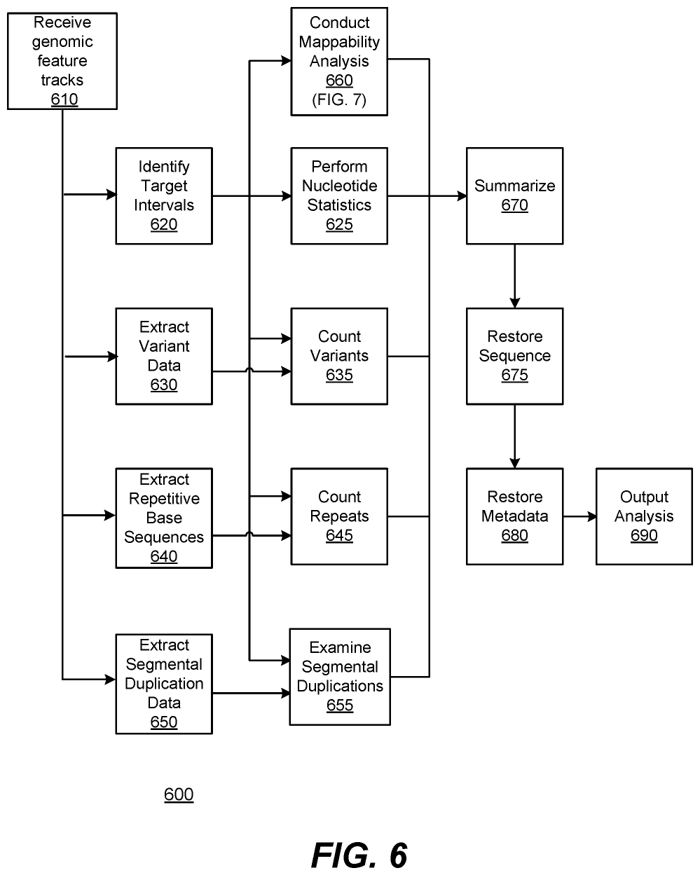

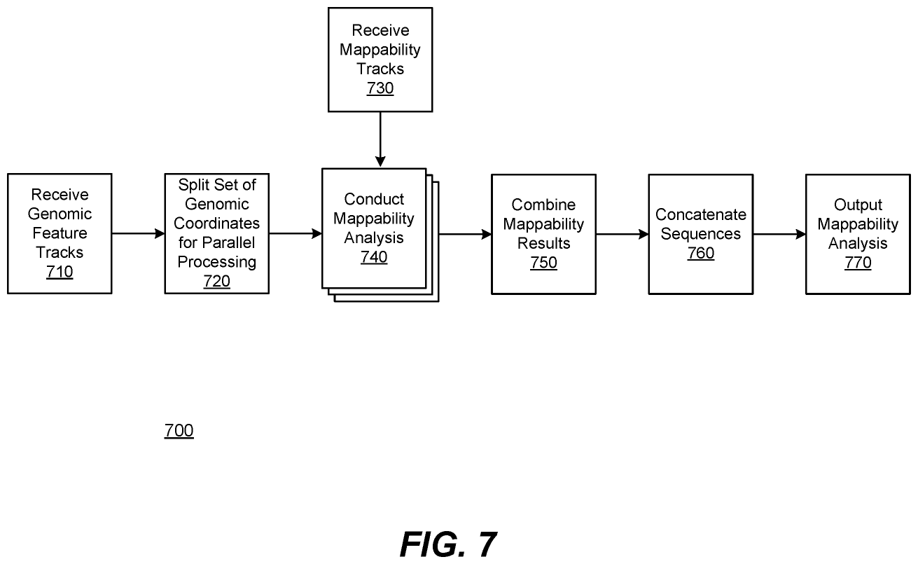

Various post-processing actions may also be performed by TIB system 130. For example, the TIB system 130 may store version information of the genomic feature tracks in one or more metadata fields of the genomic feature tracks to generate 490 versioned genomic feature tracks. The versioning information may include a particular biomarker data server's data version corresponding to a particular genomic interval included in the genomic feature tracks. The TIB system 130 may also analyze 495 the genomic coordinates in the genomic feature tracks to determine the potential performance of NGS for one or more identified genomic intervals. The details of the analyses related to the performance of NGS will be discussed with reference to FIGS. 6 and 7.

The output genomic feature tracks may be provided to the user in one or more various ways. In one embodiment, the genomic feature tracks may be transmitted to the user through emails, download links, file transfer, or other suitable ways. In another embodiment, the TIB system 130 may deposit the genomic feature tracks to data store 120, which is accessible by the user. For example, the TIB system 130 may provide an instance identifier for the user that is associated with the genomic feature tracks. The TIB system 130 may also provide one or more URIs of the data store 120 for the locations of the genomic feature tracks. The output genomic feature tracks may be stored as tab-delimited text files such as BED files.

### Example Biomarker Coordinates Generation Process

FIG. 5 is a flowchart depicting an example process 500 that generates one or more biomarker coordinates, standardized or unstandardized, in accordance with an embodiment. Example biomarker coordinates may be a set of genomic coordinates. The process 500 may be one of the workflow processes performed by the TIB system 130 using one of the nodes 228 of the computing environments 226 in interacting with one or more biomarker data servers 150. In one embodiment, the process 500 may correspond to the regeneration 435 of sets of genomic coordinates. In another embodiment, the process 500 may also correspond to the searching 440 of genomic intervals and generation 445 of standardized genomic feature tracks.

In response to a request to generate genomic feature tracks based on one or more gene identifiers 405, the TIB system 130 communicates with one or more biomarker data servers 150 (e.g., via API of the biomarker data server 150) to retrieve various information from those servers 150. The biomarker data servers 150 that provide information to the TIB system 130 may include any suitable biomarker data servers. For example, the biomarker data servers 150 may include one or more genomic coordinate databases 510 and 530, a sequence version database 520, a gene name database 540, a mutation information database 550, and a variant-phenotype relation database 560. In various embodiments, the TIB system 130 may retrieve information from additional or fewer databases. The TIB system 130 may also retrieve information from a different type of database that is not shown in FIG. 5.

A genomic coordinate database, such as the first genomic coordinate database 510 and second genomic coordinate database 530, may be a genome browser that provides and displays information related to genomic data, such as sequences and genomic coordinates. The genomic coordinate database may provide annotated genomic data including genomic coordinates, base sequences, gaps, gene prediction and structure, proteins, expression, regulation, and variation. The information may pertain to various types of regions such as protein-coding regions, non-coding RNA regions, and introns. A genomic coordinate database may provide information related to common variants and mutations in a genomic interval. A genomic coordinate database may also provide information related to correlations between different sets of genes. A genomic coordinate database may also provide information related to the connections between a phenotype and a gene so that TIB system 130 may identify diseases (or other phenotypes) related genetic coordinates specified in gene identifiers 405. A genomic coordinate database may further include information related to specific regions in the genome such as regulatory regions, promoter and control regions, repetitive regions, etc. Example genomic coordinate databases may include UCSC Genome Browser, Ensembl Genome Browser, National Center for Biotechnology Information (NCBI) Map Viewer, etc. For example, in one embodiment, the first genomic coordinate database 510 may correspond to UCSC Genome Browser and the second genomic coordinate database 530 may correspond to Ensembl Genome Browser. In various embodiments, TIB system 130 may retrieve information from any suitable number of genomic coordinate databases.

Each genomic coordinate database 510 or 530 may support different methods for data retrieval. For example, the first genomic coordinate database 510 and the second genomic coordinate database 530 may use different types of communication protocols (e.g., different API protocols or other different application layer protocols). In one embodiment, the first genomic coordinate database 510 uses RSYNC for transferring and synchronizing files. To request information from the first genomic coordinate database 510, the TIB system 130 may transmit an RSYNC call 512 that includes one or more gene identifiers 405. In response, the first genomic coordinate database 510 provides the information requested, which may be in the form of an unstandardized set of genomic coordinates. The TIB system 130 receives 514 the unstandardized set of genomic coordinates.

Some of the genomic coordinate databases may use different methods for the retrieval of gene information and genomic coordinates. For example, in one embodiment, the second genomic coordinate database 530 may include a sequence version database 520 that stores information regarding the versioning control of the sequence data in the second genomic coordinate database 530. In one embodiment, the sequence version database 520 and the second genomic coordinate database 530 may both use the File Transfer Protocol (FTP) for information exchange. The TIB system 130 may transmit an FTP call 532 that includes one or more gene identifiers 405 to the second genomic coordinate database 530. The TIB system 130 receives 534 an unstandardized set of genomic coordinates from the second genomic coordinate database 530. The TIB system 130 may use another FTP call 522 to provide information regarding the set of genomic coordinates (such as the metadata or the genomic coordinate itself) with the sequence version database 520. In turn, the TIB system 130 receives 524 the sequence version information of the set of genomic coordinates received from the second genomic coordinate database 530. Other protocols for data retrieval, such as biomaRt, are also possible for various genomic coordinate databases.

The sets of genomic coordinates generated or received from various genomic coordinate databases may not be standardized among each other such that data is presented in different ways. For example, for the same gene, different genomic coordinate databases may use different gene names or gene symbols to describe it. Even for the same gene symbols, the format of the gene symbols, such as punctuation and capitalization, may be different. In some cases, the genomic or chromosomic coordinates of the same gene may be different in terms of values and formats. The differences among different genomic coordinate databases may be present in both coding regions and non-coding regions.

Another example biomarker data server 150 may be a gene name database 540. In one embodiment, the TIB system 130 may provide one or more gene symbols to the gene name database 540 via an FTP call 542. A gene symbol may be a unique abbreviation for a gene name. The TIB system 130 receives 544 standardized gene identifiers from the gene name database 540. For example, the TIB system 130 may receive a reference file that includes standardized gene identifiers that are standardized in accordance with HGNC identifiers. The gene name database 540 may also provide information such as uniform resource identifiers (URIs) of the gene data stored in one or more genomic coordinate databases. The TIB system 130 may rely on the gene name database 540 to identify the standardized HGNC identifiers so that information regarding the same gene may be retrieved from different genomic coordinate databases.

Another example biomarker data server 150 on which the TIB system 130 may rely is a mutation information database 550. A mutation information database 550 may include published or otherwise known gene mutations that are associated with genetic diseases. The mutation information database 550 may be a collection of data of mutation entries that are extracted from scientific journals or other medical data sources. The TIB system 130 may use a Structured Query Language (SQL) call 552 to receive 554 mutation data related to one or more gene or genetic loci that are identified by the gene identifiers 405. The data provided by the mutation information database 550 may be in tabular form. The TIB system 130 may parse the mutation data and cut and merge different tables provided by the mutation information database 550.

Another example biomarker data server 150 may be a variant-phenotype relation database 560, which provides information regarding relationships among nucleotides variants (e.g., SNPs, indels) and phenotypes. The TIB system 130 may transmit an RSYNC call 562 to retrieve 564 variant data. The variant data provided by the variant-phenotype relation database 560 may be in reference sequences of variants. The variant data may be in a format such as tab-delimited subsets or variant call format (VCF). The TIB system 130 may split the received reference sequences into multiple subsets for parallel processing. The TIB system 130 may generate standard variants for the split reference sequences and concatenate the standard variants. The TIB system 130 may also filter the variants for pathogenicity.

If the TIB system 130 cannot access a biomarker data server, the TIB system 130 may source previously downloaded snapshots, genomic feature tracks, or other data that may be stored in the data store 120.

In one example embodiment, the first genomic coordinate database 510 is UCSC Genome Browser; the sequence version database 520 is Ensembl Genome Browser FTP database; the second genomic coordinate database 530 is Ensembl Genome Browser Biomart database; the gene name database 540 is GENENAMES.ORG; the mutation information database is HGMD; and the variant-phenotype relation database 560 is ClinVar. However, this particular embodiment is for illustration only. In various embodiments, one or more biomarker data servers 150 may be skipped or added. One or more biomarker data servers 150 may also be different. The biomarker data servers 150 used by the TIB system to retrieve information may include any suitable data servers that provide data related biomarkers such as genetic data, protein data, phenotype data, mutation data, variant data, medical data, scientific journals and studies, and any other suitable data.

The TIB system 130 may use HGNC reference files retrieved from the gene name database 540 to supplement 570 data for various unstandardized sets of genomic coordinates and data and standardize 580 various outputs. The standardized outputs may include genomic coordinates and other fields. One or more fields may be identifiable by HGNC. The TIB system 130 may use the HGNC reference files to fill in one or more missing HGNC fields. In standardizing the outputs, the TIB system 130 may refer to the HGNC reference files and determine one or more standardized formats and coordinates for various genes. For example, the genomic coordinates obtained from different sources may be standardized based on HGNC reference files. The formats, symbols, and identifiers of the genes may also be standardized. The TIB system 130 may also annotate intervals with, for example, the HGNC approved gene symbols, HGNC identifiers, and interval data sources. Other annotations may include RefSeq transcript, exon, number, HGMD accession, Ensembl transcript identifier, UCSC Genome Browser timestamp, HGVS variant, ClinVar identifier, and types of regions (e.g., intron, coding regions, UTR, etc.).

The TIB system 130 may combine the outputs and generate 590 one or more standardized sets of genomic coordinates, which may be included in the form of genomic feature tracks. For example, the mutation and variant data may be combined with the genomic coordinates that are retrieved from the genomic coordinate databases 510 and 530 in generating a set of genomic feature tracks. Examples of genomic feature tracks are shown in FIGS. 8A and 8B and will be discussed in further detail. In generating genomic feature tracks, the TIB system 130 may sort one or more genomic coordinates generated from different biomarker data servers 150, deconvolute those genomic coordinates, parse standard genomic coordinates to data fields in genomic feature tracks, expand the exon regions by adding a base range to both ends of a gene, and sort genomic feature tracks for exon calling. The standardized genomic feature tracks may be formatted as one or more BED files as outputs. Multiple sets of genomic feature tracks may be combined and sorted to generate a combined standardized genomic feature tracks. The combined standardized genomic feature tracks may be referred to as a TIB Full BED file and may be used as a result when a user selects the option “Regenerate TIB BED” 340 in FIG. 3.

### Example Amenability Analyses for Massively Parallel Sequencing

FIG. 6 is a flowchart depicting an example process 600 of performing amenability analyses for massively parallel sequencing (e.g., NGS), in accordance with an embodiment. The process 600 may use information of standardized genomic feature tracks to predict how likely a region identified by a gene identifier 405 is going to be successfully sequenced using a massively parallel sequencing technique. Using the processes 400 and 500, the TIB system 130 may collect and aggregate the information related to a particular gene identifier 405 to generate one or more standardized genomic feature tracks. In turn, the data in the genomic feature tracks may be used to predict the likelihood of success in sequencing regions in genomic coordinates of interest. The TIB system 130 automatically analyzes the genomic feature tracks to gather metrics for parameters that are known to impact sequencing performance, such as segmental duplications, repeats, and uniqueness of read alignment.

By way of example, the TIB system 130 may receive 610 one or more input sets of genomic feature tracks. Each set may be formatted as a BED file. The genomic feature tracks may correspond to the outputs generated in 485 of FIG. 4, the outputs generated in 590 of FIG. 5, or any other suitable genomic feature tracks or annotated sets of genomic coordinates. The TIB system 130 identifies 620 target intervals included in the genomic feature tracks. For example, one or more target intervals may be extracted or identified based on the process 400 and/or the process 500. A user may specify a target interval for determining the feasibility to conduct massively parallel sequencing. The target intervals may be merged or un-merged and may include coding or non-coding regions. The target intervals may be expressed in standardized genomic coordinates and including additional flanking regions based on the user's request. Information regarding the target intervals may be retrieved from one or more biomarker data servers 150 and standardized, as discussed in process 500.

The TIB system 130 may generate various forms of statistics on the information of the target intervals. For example, the TIB system 130 may perform 625 nucleotide statistics to determine the composition of nucleotides such as the numbers of A, T, C, and G in the target intervals. In one embodiment, the TIB system 130 uses bedtools Nuc to determine the composition of nucleotides. The nucleotide statistics may provide the degree of uniqueness of a particular sequence of nucleotides, such as a read alignment, in a target interval. Sequencing is more likely to be successful for a more unique sequence. The nucleotide statistics may also identify one or more sequences that are prone to a failure or a success in sequencing. The TIB system 130 may also determine the ratio of GC content to AT content in a target interval or a sequence in the interval. An interval with a high GC ratio often makes a probe in the sequencing harder to hybridize. Other suitable statistics may also be possible.

The TIB system 130 may extract 630 variant data from the information included in the genomic feature tracks or from another source. For example, the variant data may be obtained from HGMD. Another example of statistics on the information of the target intervals may include counting 635 the number of potential variants that fall within the target intervals. The count of potential variants may be used in determining the need to use an orthogonal method, in one embodiment, Sanger sequencing, to fill in gaps that are not amenable to NGS.

The TIB system 130 may also extract 640 repetitive base sequences in the target intervals. For example, a target interval may include a long segment of a repetitive GC sequence. The TIB system 130 may count 645 the repeats and generate statistics regarding the repeats in the target intervals. Other types of statistics on the repeats may also be performed. The information of repetitive base sequences in a target interval may be included in genomic feature tracks or may be obtained from another source, such as a UCSC Repeats BED file. Massively parallel sequencing techniques often generate short read lengths and high data volumes. Repeats present technical challenges for sequence alignment to a reference genome because repeats create ambiguities in alignment and assembly.

The TIB system 130 may also extract 650 segmental duplication data in the target intervals. For example, the data may be extracted from the genomic feature tracks or from another source such as UCSC segmental duplication database. Segmental duplications are segments of DNA sequences that have similar copies in other regions of the genome. Segmental duplications may present challenges in the sequence alignment to a reference genome because of the ambiguity the duplicative segments cause in determining the precise location of a sequence result. The TIB system 130 may examine 655 segmental duplications that are present in a target interval to evaluate the likelihood of success in sequencing the target interval.

The TIB system 130 may also perform other suitable statistics on the information of the target intervals to generate additional metrics that may be used to evaluate the amenability of massively parallel sequencing. The TIB system 130 may also perform mappability analysis 660, which will be discussed with reference to FIG. 7.

Based on one or more metrics determined in, for example, steps 625, 635, 645, and 655, the TIB system 130 may summarize 670 the result that determines the likelihood of success of sequencing of the identified genomic interval using a massively parallel sequencing technique. The summary may include an aggregated likelihood that weighs the various metrics or may include a breakdown of various metrics in affecting the likelihood of success. A report may also be generated that includes the summary and some key issues that may affect the performance of the sequencing. The TIB system 130 may also propose other intervals for sequencing. The report may be included in the genomic feature tracks that will be generated as output of process 600.

The TIB system 130 may restore 675 the sequence and restore 680 the metadata in the genomic feature tracks based on the inclusion of new information generated from the amenability analysis. The TIB system 130 may reformat the BED file to various standard formats as needed for various sub-analyses. For example, some analyses may require the BED file to have no headers, typically delineated using at the start of the line. The TIB system 130 may return headers that were removed for these processes. Some processes require BED coordinates to start with “chr1,” other with just “1.” The TIB system 130 may return “chr” to the beginning of intervals in which the prefix has been removed. The TIB system 130 outputs 690 the analysis to the user by transmitting the output to the user or depositing the output to the data store 120.

### Example Mappability Analysis

FIG. 7 is a flowchart depicting an example process 700 of performing mappability analyses for massively parallel sequencing, in accordance with an embodiment. The TIB system 130 may receive 710 input genomic feature tracks and identify one or more target intervals. The TIB system 130 may split 720 genomic coordinates included in the input genomic feature tracks into a plurality of subsets of genomic coordinates. In one embodiment, the set of genomic coordinates is split into smaller files (e.g., smaller BED files) for each chromosome. The TIB system 130 may perform the splitting 720 for parallel processing. Processing the files in parallel may dramatically reduce the computation time. The TIB system 130 may receive 730 a mappability tracks. For example, the mappability tracks may include a reference genomic sequence for the target intervals. The mappability analysis may be obtained from the ENCODE project.

The TIB system 130 may conduct 740 a mappability analysis for each subset of genomic coordinates. For example, for each of the plurality of split genomic coordinates, the TIB system 130 may compare coordinates in the subset to one or more reference genomes to determine how often sequences of some length (e.g., 100 base sequencing reads) align uniquely with the coordinates of target intervals in the set of genomic coordinates. The analysis may be expressed as a metric that measures the mappability. For example, 100 base sequence reads may be equally likely to align to the region of the candidate target interval as to another region. This may result in a score of the metric 0.5. The mappability metric predicts the difficulties in a sequencing process because the true location in the genome of a variant that falls into a poorly mapped region may be difficult to determine. For each of the plurality of subsets, the TIB system may generate a mappability metric based on how often sequences align uniquely with the coordinates in the subset. In determining the mappability, the TIB system 130 may take into account of one or more metrics determined in the process 600 that are relevant to the short sequence, including, for example, the number of variants, the GC content, the repeats, and the segmental duplications in the short sequence.

The TIB system 130 may combine 750 the mappability metrics in various subsets to generate a joined mappability metric for the set of genomic coordinates. The TIB system 130 may concatenate 760 the short sequences to regenerate the sequence for the candidate target interval. The TIB system 130 may output 770 the mappability analysis. In some embodiments, the mappability analysis may be included in the output of the sequencing amenability analysis as illustrated in process 600. For example, the mappability analysis may be added to genomic feature tracks, such as a merged standardized set of genomic feature tracks. In other embodiments, the mappability analysis may be output as a separate report.

### Example Standardized Sets of Genomic Feature Tracks

FIGS. 8A and 8B are conceptual diagrams illustrating various examples of different versions of genomic feature tracks, in accordance with some embodiments. The standardized genomic feature tracks output by the TIB system 130 may take various suitable forms, which may include an unmerged standardized genomic feature tracks 800 shown in FIG. 8A and a merged standardized genomic feature tracks 850 shown in FIG. 8B. A set of genomic feature tracks may be in any suitable file formats, such as in a tabular form that may be saved as a spreadsheet file, a comma-separated values (CSV), tab-delimited file, HTML, XML, PDF, JSON, etc. In one embodiment, a set of genomic feature tracks is saved as a tab-delimited text file such as a BED file.

The standardized genomic feature tracks 800 may include a header section 810 and a body section 820. The header section 810 may list versioning information, metadata, and information regarding files that are used to compile the standardized genomic feature tracks 800. For example, the TIB system 130, based on one or more input files, may use one or more of the processes 400, 500, 600, and 700 to retrieve files from various biomarker data servers 150. Both the input files and the files retrieved from the biomarker data servers 150 may be listed in the header section 810. Each of the files listed in the header section 810 may also be hashed to generate a unique hash identifier of the file. For example, a file may be passed through an MD5 hash algorithm to generate the file's hash identifier. The header section 810 may also include the metadata of the files such as the sources of the files, types of the files, version information of the files, etc. The hash identifier allows the TIB system 130 and any users to verify one or more files because a change in the contents of a file will change the hash identifier.

The body section 820 includes the data of the standardized genomic feature tracks 800. The data may be provided in a tabular form that includes multiple columns. In FIG. 8A, the titles of each column are shown. Although the titles are separated in two lines in FIG. 8A for illustration purpose, the titles may correspond to a single row in the body section 820. For each genomic interval, the genomic feature tracks 800 may include the chromosome number 822, start coordinate 824, stop coordinate 826, the custom name for interval 828, HGNC identifier 830, MIM identifier 832, transcript identifier 834, exon information 836, accession 838, Human Genome Variation Society (HGVS) coding DNA reference sequence (C_DOT) 840, type 842, source 844, original start 846, and original end 848. In various embodiments, a genomic interval may include additional or less information. A genomic interval may also include different information that is not listed as a column in FIG. 8A. The body section 820 may include information regarding one or more target intervals.

The chromosome number 822, the start coordinate 824, and the end coordinate 826 may collectively define a genomic interval. The start coordinate 824 and the end coordinate 826 may correspond to adjusted start and end coordinates that represent an expanded interval. For example, in FIG. 3, a user may select a range of exon expand 350. The user may also upload an include BED file that contains coordinates that should be included in the genomic feature tracks 800. The start coordinate 824 and the end coordinate 826 may reflect the various expansions that are selected by the user or the TIB system 130.

The custom name for interval 828 may be a customized name that is provided by the TIB system 130. In one embodiment, the custom name for interval 828 may be the fourth column of the standardized genomic feature tracks. In some cases, some software applications for BED files may truncate data that are beyond the fourth column. In one embodiment, the custom name for interval 828 may include the data for the some of the latter columns combined. For example, in one case, a custom name for an example interval may be NBPF20_HGNC:32000_NM_001278267._1_UCSC_20190325_123655_847864_NU LL_5PUTR_TRANSCRIPT, which may include the data values for the columns 830 through 844.

The HGNC identifier 830 provides the standardized HGNC identifier of the genomic interval. The MIM identifier 832, or OMIM identifier, provides the OMIM identifier related to the genes described by the genomic interval. The transcript identifier 834 identifies whether an NM or an NR transcript is described by the genomic interval. The exon information 836 provides the exons that are included in the genomic interval. Accession 838 provides data related to either an Ensembl identifier or a UCSC timestamp that is relevant to the genomic interval. C_DOT 840 identifies variants in the notation of HGVS coding DNA reference sequence. The type column 842 identifies the type of genomic interval, such as whether the interval is CDS, UTR, or another type of interval, coding or non-coding. The source column 844 identifies the source of the information related to the genomic interval. For example, a source may be a biomarker database, HGMD, etc. The original start 846 and the original end 848 list the coordinates of the genomic interval without any expansion, merging, etc.

Depending on the input gene identifiers provided by the user, the genomic feature tracks 800 includes more than one genomic intervals that are relevant to the inputs. Each of the genomic intervals found may be listed as a row of the body section 820.

FIG. 8B illustrates another example of a set of genomic feature tracks, which may take the form of a merged standardized genomic feature tracks 850. The merged standardized genomic feature tracks 850 may also include the header section 855 and the body section 860. The header section 855 is similar to the header section 810 discussed in FIG. 8B. The body section 860 is illustrated by the titles of the columns, which include genomic coordinates 862, feature 864, HGMD count 866, HGMD variants, 868, base-pair statistics 870, sequence length 872, repeats count 874, segmental duplication count 876, segmental duplication location 878, mappability of target interval based on a first length of a sequence 880, mappability of target interval based on the second length 882, and the average mappability 884. In various embodiments, a genomic interval may include additional or less information. A genomic interval may also include different information that is not listed as a column in FIG. 8B. The body section 860 may include information regarding one or more target intervals.

The merged genomic feature tracks 850 may include the interval information that is similar to the standardized genomic feature tracks 800, information derived from amenability analyses for massively parallel sequencing as described in process 600 and from the mappability analysis as described in process 700, and other suitable information such as mutation and variant data. For example, in one embodiment, the genomic coordinates 862 may include three columns that specify the chromosome number, the adjusted start coordinate, and the adjusted end coordinate. The feature 864 may be the fourth column of the genomic feature tracks 850. The feature 864 may include the interval information in a condensed form similar to the custom name for interval 828. In one embodiment, the feature 864 may include HGNC identifier 830, MIM identifier 832, transcript identifier 834, exon information 836, accession 838, HGVS coding DNA reference sequence (C_DOT) 840, type 842, and source 844 combined as a string. An example feature 864 may be: NBPF20_HGNC:32000_NM_001278267._1_UCSC_20190325_123655_847864_NU LL_5PUTR_TRANSCRIPT.

The HGMD count 866 includes the number of potential mutations in a target interval. The HGMD variant 868 provides the variant information. The base pair statistics 870 includes one or more columns of statistical values regarding the nucleotides in the target interval. For example, the columns may include AT content percentage, GC content percentage, number of A, number of T, number of G, and the number of other nucleotides (e.g., imputed or unidentified nucleotides). The base pair statistics 870 provides insight as to the likelihood of a successful sequencing for the target interval. For example, a high GC content percentage in a target interval may result in the probes being more difficult to hybridize during the sequencing. The sequence length 872 provides information regarding the length of the target interval.

The repeats count 874 provides the number of repeats in the target interval. The segmental duplication count 876 provides the number of segmental duplications in the target interval. The segmental duplication locations 878 provides the coordinates of the segmental duplication in other genetic loci (e.g., in the same chromosome or other chromosomes).

The genomic feature tracks 850 may also include the mappability analyses for one or more target intervals. For example, the mappability may depend on the length of a typical segment in a massively parallel sequencing. A segment with a longer length is typically easier to map to a reference genome, but it is usually more expensive to sequence and may not be possible with certain sequencing instruments. The genomic feature tracks 850 may include the mappability metrics for one or more lengths, such as the mappability metric 880 for a first length and the mappability metric 882 for a second length. The genomic feature tracks 850 may also provide the average mappability 884 of the target interval.

While two example versions of standardized genomic feature tracks 800 and 850 are shown, other standardized genomic feature tracks in various embodiments may be in different formats and contains different information and data. For example, some standardized genomic feature tracks may not need to include the header section. A set of standardized genomic feature tracks may standardize the format and notation of genomic coordinates and related annotation. The precise format and data fields of a set of standardized genomic feature tracks may depend on implementation and the decision of the operator of TIB system 130.

### Example Processes

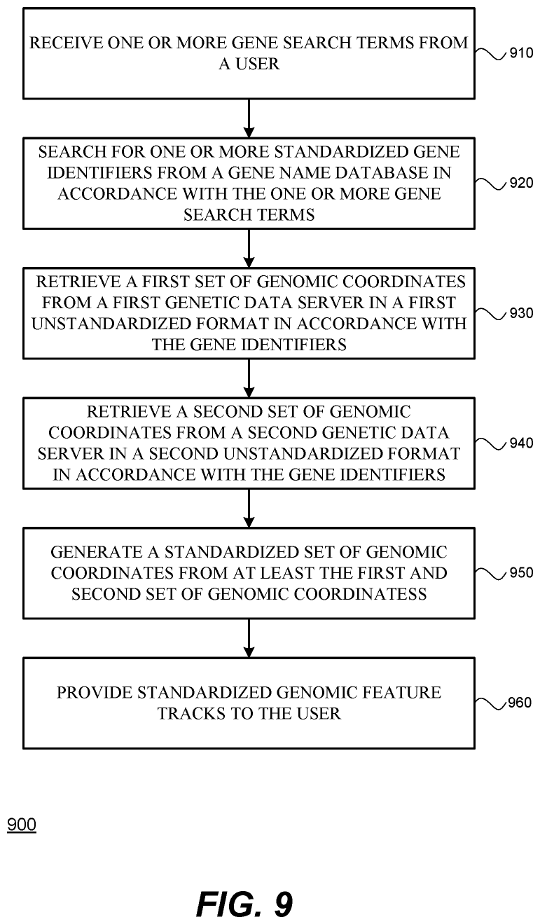

FIG. 9 is a flowchart depicting an example process 900 of generating standardized genomic feature tracks, in accordance with an embodiment. The example process 900 may be performed by the TIB system 130. The TIB system 130 receives 910 one or more gene search terms from a user. The gene search terms may be unstandardized gene names, standardized gene identifiers, or other suitable terms. The TIB system 130 searches 920 for one or more standardized gene identifiers from a standardized reference set of genomic coordinates in accordance with the one or more gene search terms. In some cases, the gene search terms provided by the users may directly be gene identifiers. The TIB system 130 may still validate the gene identifiers based on the data retrieved from the gene name database, such as the HGNC.

The TIB system 130 retrieves 930 a first set of genomic coordinates from a first biomarker data server 150 in accordance with the gene identifiers. The first set of genomic coordinates may be in a first unstandardized format. The first set of genomic coordinates may be associated with information of a first set of one or more genomic intervals that are identified from the gene identifiers. Likewise, the TIB system 130 retrieves 940 a second set of genomic coordinates from a second biomarker data server 150 in accordance with the gene identifiers. The second set of genomic coordinates may be in a second unstandardized format. The second set of genomic coordinates may be associated with information of a second set of one or more genomic intervals that are identified from the gene identifiers.

The TIB system 130 generates 950 a standardized set of genomic coordinates from at least the first set of genomic coordinates and the second set of genomic coordinates. The standardized set of genomic coordinates includes genomic coordinates of target intervals derived from the genomic intervals in the first set and the second set. For example, a target interval may be derived from one of the genomic intervals with flanking of both ends. Another target interval may be derived from merging two or more genomic intervals (which may be retrieved from the same or different sources). The standardized set of genomic coordinates may express the target intervals in a standardized format. In generating a standardized set of genomic coordinates, the TIB system 130 may annotate each target interval with various information as shown in example columns of FIGS. 8A and 8B. Example details of the retrieval of sets of genomic coordinates and generation of a standardized set of genomic coordinates are discussed with reference to FIGS. 4 and 5. The collection of information may be generated as standardized genomic feature tracks. Examples of standardized genomic feature tracks are illustrated in FIGS. 8A and 8B.

The TIB system 130 provides 960 the standardized genomic feature tracks to the user. For example, the TIB system 130 may directly transmit data of the standardized genomic feature tracks to a user device for display of the annotated set of genomic coordinates. The TIB system 130 may also transmit the standardized genomic feature tracks by email, file sharing, downloading, etc. The TIB system 130 may also upload the standardized genomic feature tracks to a data store 120, which is accessible by the user.

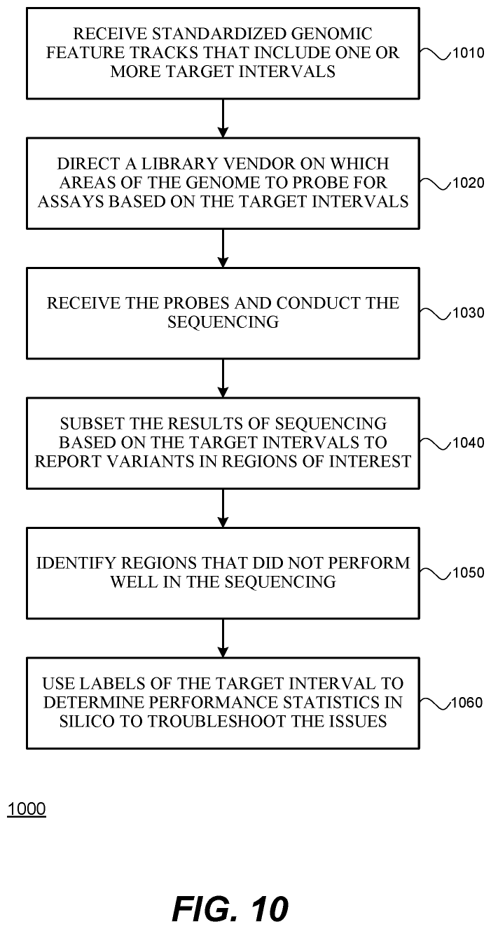

FIG. 10 is a flowchart depicting an example process 1000 of performing biological sample analyses that involve massively parallel sequencing, in accordance with an embodiment. In a massively parallel sequencing, a hybridization capture-based target enrichment uses custom-designed oligonucleotide probes to capture specific regions of the genome. In one embodiment, a TIB system 130 performs the computational steps specifying target intervals for a given list of gene identifiers. The system may create sets of genomic feature tracks (e.g., in the format of BED files) that include annotations for known/potential disease-causing variants from HGMD, and ClinVar, as well as NCBI RefSeq transcripts (exons, introns, CDS, UTR). The genomic feature tracks may be versioned. A user receives 1010, from the TIB system 130, one or more genomic feature tracks that include one or more target intervals.

The genomic feature tracks can be provided to a custom capture library vendor for the design of probes for NGS capture libraries, and may also be used for downstream masking of clinically excluded genes or regions during NGS data analysis. The TIB system 130 annotates genomic intervals to facilitate the design and filtering of targeted NGS capture libraries. Standardized annotation retains interval provenance throughout library design. The user directs 1020 a library vendor on which areas of the genome to probe for assays based on the target intervals. For example, custom-designed oligonucleotide probes may be designed based on the target intervals. The user receives 1030 the probes and conducts the sequencing. The user subsets 1040 the results of sequencing based on the target intervals to report variants in regions of interest. For example, sequencing data may be subset at runtime by filtering annotated intervals against a custom list of HGNC identifiers. The user identifies 1050 regions that did not perform well in the sequencing. The user uses 1060 labels of the target interval to request the TIB system 130 to determine performance statistics in silico to troubleshoot the issues, for example, using processes 600 and 700. The process 1000 may also be performed by any other suitable entities such as the entity that operates the TIB system 130.

### Computing Machine Architecture

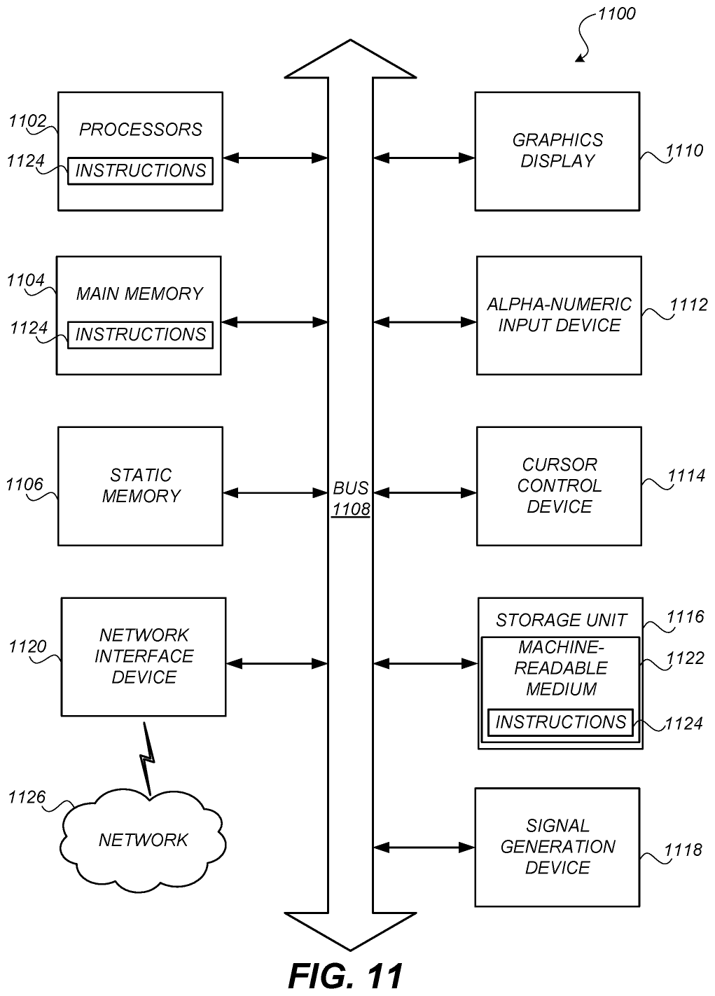

FIG. 11 is a block diagram illustrating components of an example computing machine that is capable of reading instructions from a computer-readable medium and execute them in a processor (or controller). A computer described herein may include a single computing machine shown in FIG. 11, a virtual machine, a distributed computing system that includes multiples nodes of computing machines shown in FIG. 11, or any other suitable arrangement of computing devices.

By way of example, FIG. 11 shows a diagrammatic representation of a computing machine in the example form of a computer system 1100 within which instructions 1124 (e.g., software, program code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 11 may correspond to any software, hardware, or combined components shown in FIGS. 1 and 2, including but not limited to, the user device 110, the TIB system 130, the biomarker data servers 150, and various engines, modules, interfaces, terminals, computing node 228 and machines shown in FIG. 2. While FIG. 11 shows various hardware and software elements, each of the components described in FIGS. 1 and 2 may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 1124 that specify actions to be taken by that machine. Further, while only a single machine is illustrated, the term “machine” and “computer” may also be taken to include any collection of machines that individually or jointly execute instructions 1124 to perform any one or more of the methodologies discussed herein.

The example computer system 1100 includes one or more processors 1102 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 1100 may also include a memory 1104 that store computer code including instructions 1124 that may cause the processors 1102 to perform certain actions when the instructions are executed, directly or indirectly by the processors 1102. Instructions can be any directions, commands, or orders that may be stored in different forms, such as equipment-readable instructions, programming instructions including source code, and other communication signals and orders. Instructions may be used in a general sense and are not limited to machine-readable codes. The processors 1102 may include one or more multiply-accumulate units (MAC units) that are used to perform computations of one or more processes described herein.

One and more methods described herein improve the operation speed of the processors 1102 and reduces the space required for the memory 1104. For example, the various processes described herein reduce the complexity of the computation of the processors 1102 by applying one or more novel techniques that simplify the steps in analyzing data and generating results of the processors 1102. The algorithms described herein also reduces the size of the models and datasets to reduce the storage space requirement for memory 1104.

The performance of certain of the operations may be distributed among the more than processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations. Even though in the specification or the claims may refer some processes to be performed by a processor, this should be construed to include a joint operation of multiple distributed processors.

The computer system 1100 may include a main memory 1104, and a static memory 1106, which are configured to communicate with each other via a bus 1108. The computer system 1100 may further include a graphics display unit 1110 (e.g., a plasma display panel (PDP), a liquid crystal display (LCD), a projector, or a cathode ray tube (CRT)). The graphics display unit 1110, controlled by the processors 1102, displays a graphical user interface (GUI) to display one or more results and data generated by the processes described herein. The computer system 1100 may also include alphanumeric input device 1112 (e.g., a keyboard), a cursor control device 1114 (e.g., a mouse, a trackball, a joystick, a motion sensor, or other pointing instrument), a storage unit 1116 (a hard drive, a solid state drive, a hybrid drive, a memory disk, etc.), a signal generation device 1118 (e.g., a speaker), and a network interface device 1120, which also are configured to communicate via the bus 1108.

The storage unit 1116 includes a computer-readable medium 1122 on which is stored instructions 1124 embodying any one or more of the methodologies or functions described herein. The instructions 1124 may also reside, completely or at least partially, within the main memory 1104 or within the processor 1102 (e.g., within a processor's cache memory) during execution thereof by the computer system 1100, the main memory 1104 and the processor 1102 also constituting computer-readable media. The instructions 1124 may be transmitted or received over a network 1126 via the network interface device 1120.

While computer-readable medium 1122 is shown in an example embodiment to be a single medium, the term “computer-readable medium” should be taken to include a single medium or multiple media (e.g., a centralized or distributed database, or associated caches and servers) able to store instructions (e.g., instructions 1124). The computer-readable medium may include any medium that is capable of storing instructions (e.g., instructions 1124) for execution by the processors (e.g., processors 1102) and that causes the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

In various embodiments, a non-transitory computer readable medium that is configured to store instructions may be used. The instructions, when executed by one or more processors, cause the one or more processors to perform steps described in the above computer-implemented processes or described in any embodiments of this disclosure. In various embodiments, a system may include one or more processors and a storage medium that is configured to store instructions. The instructions, when executed by one or more processors, cause the one or more processors to perform steps described in the above computer-implemented processes or described in any embodiments of this disclosure.

### Additional Considerations

Beneficially, various processes and systems described herein allow researchers and scientists to dramatically expedite the review of potential problems in the quality of raw sequencing results and data in NGS. A TIB system may be used to generate target intervals to direct sequencing vendors on which areas of the genome to probe for assays. The TIB system allows the provenance of the coordinates of the genes, variants, and other genomic regions of interest to be traced to their source, which promotes accuracy of variant identification, classification, and reporting in NGS. After the probes are generated, the target intervals may be used to subset the results of sequencing to report variants in regions of interest. When regions do not perform, the labels of the target intervals and the associated data may use in silico performance statistics to troubleshoot the issues. For example, researchers and scientists may observe high GC content or poor mappability in an interval, if the interval fails sequencing. The processes described herein improve the quality and results of NGS.

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Any feature mentioned in one claim category, e.g. method, can be claimed in another claim category, e.g. computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject-matter may include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein may be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines may be embodied in software, firmware, hardware, or any combinations thereof.

Any of the steps, operations, or processes described herein may be performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure may describe a process that includes multiple steps sequentially with arrows present in a flowchart, the steps in the process do not need to be performed by the specific order claimed or described in the disclosure. Some steps may be performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not always imply that all members are associated with an element A. Instead, the term “each” only implies that a member (of some of the members), in a singular form, is associated with an element A. In claims, the use of a singular form of a noun may imply at least one element even though a plural form is not used.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

What is claimed is:

1. A computer-implemented method, comprising:
receiving, by a target interval builder system, one or more gene identifiers from a user device of a user;
retrieving, by the target interval builder system, a first set of genomic coordinates from a first biomarker data server, the first set of genomic coordinates associate with information of a first set of one or more genomic intervals that are identified from the one or more gene identifiers, the first set of genomic coordinates in a first unstandardized format;
retrieving, by the target interval builder system, a second set of genomic coordinates from a second biomarker data server, the second set of genomic coordinates associated with information of a second set of one or more genomic intervals that are identified from the one or more gene identifiers, the second set of genomic coordinates in a second unstandardized format;
generating, by the target interval builder system, a standardized set of genomic coordinates from at least the first and second sets of genomic coordinates, the standardized set of genomic coordinates comprising genomic coordinates of target intervals derived from at least some of the genomic intervals in the first set and the second set, the genomic coordinates in a standardized format; and
generating standardized genomic feature tracks from the standardized set of genomic coordinates;
providing, by the target interval builder system, the standardized genomic feature tracks to the user device of the user.

2. The computer-implemented method of claim 1, further comprising:
receiving one or more exclude genomic intervals, each exclude genomic interval intended to be excluded by the user;
identifying, in the standardized set of genomic coordinates, one or more target intervals that match the exclude genomic intervals; and
removing the identified one or more target intervals from the standardized set of genomic coordinates.

3. The computer-implemented method of claim 1, further comprising:
receiving one or more include genomic intervals, each include genomic interval intended to be included by the user;
identifying one or more additional genomic intervals that match the include genomic intervals but are not identified by the gene identifiers; and
adding the identified one or more additional genomic intervals to the standardized set of genomic coordinates.

4. The computer-implemented method of claim 1, further comprising:
splitting the standardized set of genomic coordinates into a plurality of subsets of genomic coordinates;
comparing, for each of the plurality of subsets of genomic coordinates, the genomic coordinates to one or more reference genomes to determine how often sequences align uniquely with the genomic coordinates in the subset;
generating, for each of the plurality of subsets of genomic coordinates, a mappability metric based on how often sequences align uniquely with the genomic coordinates in the subset; and
combining the mappability metrics to generate a joined mappability metric for the standardized set of genomic coordinates.

5. The computer-implemented method of claim 1, further comprising:
identifying one of the target intervals in the standardized genomic feature tracks;
generating statistics of the identified genomic interval, the statistics including one or more of the following: compositions of nucleotides, a count of potential variant sites, a count of repetitive nucleotide patterns, or a degree of segmental duplication of the identified genomic interval; and
determining, based on the statistics, a likelihood of success of sequencing of the identified genomic interval using a massively parallel sequencing.

6. The computer-implemented method of claim 1, wherein generating the standardized set of genomic coordinates from at least the first and second sets of genomic coordinates comprises:
flanking a genomic interval with additional bases at each end of the genomic interval to generate a flanked target genomic interval, the genomic interval selected from the first set or the second set.

7. The computer-implemented method of claim 1, wherein generating the standardized set of genomic coordinates from at least the first and second sets of genomic coordinates comprises:
identifying a plurality of overlapped genomic intervals, each of the plurality of overlapped genomic intervals identified from the first set or the second set; and
merging the overlapped genomic intervals to generate a merged interval, the merged interval being one of the target intervals.

8. The computer-implemented method of claim 1, wherein the standardized genomic feature tracks are versioned, a version indicating a particular biomarker data server's data version corresponding to a particular genomic interval included in the standardized genomic feature tracks.

9. The computer-implemented method of claim 1, wherein the one or more gene identifiers are standardized gene identifiers that are standardized in accordance with HUGO Gene Nomenclature Committee (HGNC).

10. The computer-implemented method of claim 1, wherein the first biomarker data server and the second biomarker data server are selected from two of the following: UCSC Genome Browser, Ensembl Genome Browser, Human Gene Mutation Database (HGMD), or ClinVar.

11. The computer-implemented method of claim 1, wherein a set of the standardized genomic feature tracks is stored as a tab-delimited text file.

12. The computer-implemented method of claim 1, wherein the standardized genomic feature tracks correspond to a merged genomic feature tracks that comprises:
genomic coordinates;
mutation site and variant site count;
nucleotide statistics; and
mappability data.

13. The computer-implemented method of claim 1, wherein the first set of one or more genomic intervals are retrieved from the first biomarker data server using a first data retrieval protocol and the second set of one or more genomic intervals are retrieved from the second biomarker data server using a second data retrieval protocol different from the first data retrieval protocol.

14. A non-transitory computer readable medium for storing computer code comprising instructions, the instructions, when executed by one or more processors, cause the one or more processors to perform steps comprising:
receiving one or more gene identifiers from a user device of a user;
retrieving a first set of genomic coordinates from a first biomarker data server, the first set of genomic coordinates comprising information of a first set of one or more genomic intervals that are identified from the one or more gene identifiers, the first set of genomic coordinates in a first unstandardized format;
retrieving a second set of genomic coordinates from a second biomarker data server, the second set of genomic coordinates comprising information of a second set of one or more genomic intervals that are identified from the one or more gene identifiers, the second set of genomic coordinates in a second unstandardized format;
generating a standardized set of genomic coordinates from at least the first and second sets of genomic coordinates, the standardized set of genomic coordinates comprising genomic coordinates of target intervals derived from the genomic intervals in the first set and the second set, the genomic coordinates in a standardized format; and
generating standardized genomic feature tracks from the standardized set of genomic coordinates;
providing, by the target interval builder system, the standardized genomic feature tracks to the user device of the user.

15. The non-transitory computer readable medium of claim 14, wherein the instructions, when executed by the one or more processors, cause the one or more processors to perform additional steps comprising:
splitting the standardized set of genomic coordinates into a plurality of subsets of genomic coordinates;
comparing, for each of the plurality of subsets of genomic coordinates, the genomic coordinates to one or more reference genomes to determine how often sequences align uniquely with the genomic coordinates in the subset;
generating, for each of the plurality of subsets of genomic coordinates, a mappability metric based on how often sequences align uniquely with the genomic coordinates in the subset; and
combining the mappability metrics to generate a joined mappability metric for the standardized set of genomic coordinates.

16. The non-transitory computer readable medium of claim 14, wherein the instructions, when executed by the one or more processors, cause the one or more processors to perform additional steps comprising:
identifying one of the target intervals in the standardized genomic feature tracks;
generating statistics of the identified genomic interval, the statistics including one or more of the following: compositions of nucleotides, a count of potential variant sites, a count of repetitive nucleotide patterns, and a degree of segmental duplication of the identified genomic interval; and
determining, based on the statistics, a likelihood of success of sequencing of the identified genomic interval using a massively parallel sequencing.

17. The non-transitory computer readable medium of claim 14, wherein generating the standardized set of genomic coordinates from at least the first and second sets of genomic coordinates comprises:
flanking a genomic interval with additional bases at each end of the genomic interval to generate a flanked target genomic interval, the genomic interval selected from the first set or the second set.

18. A system comprising:
a user interface configured to receive one or more gene identifiers from a user device of a user;
a target interval builder system storing instructions of a workflow, the instructions, when executed by one or more processors, cause the one or more processors to perform steps comprising:
retrieving a first set of genomic coordinates from a first biomarker data server, the first set of genomic coordinates comprising information of a first set of one or more genomic intervals that are identified from the one or more gene identifiers, the first set of genomic coordinates in a first unstandardized format;
retrieving a second set of genomic coordinates from a second biomarker data server, the second set of genomic coordinates comprising information of a second set of one or more genomic intervals that are identified from the one or more gene identifiers, the second set of genomic coordinates in a second unstandardized format;
generating a standardized set of genomic coordinates from at least the first and second sets of genomic coordinates, the standardized set of genomic coordinates comprising genomic coordinates of target intervals derived from the genomic intervals in the first set and the second set, the genomic coordinates in a standardized format; and
generating standardized genomic feature tracks from the standardized set of genomic coordinates;
providing, by the target interval builder system, the standardized genomic feature tracks to the user device of the user.

19. The system of claim 18, further comprising:
a cloud data store configured to store the generated standardized genomic feature tracks provided by the target interval builder system and generate one or more links corresponding to the standardized genomic feature tracks, the link accessible by the user.

20. The system of claim 18, wherein the target interval builder system is associated with a distributed computing system comprising a plurality of nodes, and a first node configured to execute the instructions to generate the first set of genomic coordinates and a second node configured to execute the instructions to generate the second set of genomic coordinates.
